(19) United States
(12) Patent Application Publication
(10) Pub. No.: US 2006/0229277 A1
(43) Pub. Date: Oct. 12, 2006

(54) Stabilized pharmaceutical compositions comprising an HMG-CoA reductase inhibitor

(75) Inventor: Laxminarayan Joshi, Toronto (CA)

Correspondence Address: PEACOCK MYERS, P.C., 201 THIRD STREET, N.W., SUITE 1340, ALBUQUERQUE, NM, 87102, US

(73) Assignee: Orbus Pharma, Inc., Markham (CA)

(21) Appl. No.: 11/102,347

(22) Filed: Apr. 8, 2005

(51) Int. Cl. A61K 31/724; A61K 31/401; A61K 31/366; A61K 31/22

(52) U.S. Cl. 514/58; 514/460; 514/548; 514/423

(57) ABSTRACT

The present invention relates to pharmaceutical compositions containing an HMG-CoA reductase inhibitor which are protected from destabilization in acidic environments by utilizing an inclusion complexing agent, and further relates to their preparation and to their use in the treatment of hypercholesterolemia and hyperlipidemia.

### BACKGROUND OF THE INVENTION

1. Field of the Invention (Technical Field)

The present invention is a new stable pharmaceutical composition that is suitable for use as an antihypercholesterolemic or antihyperlipidemia agent, and more particularly, a stable pharmaceutical composition containing as an active substance an HMG-CoA reductase inhibitor.

2. Description of Related Art

Fluvastatin, lovastatin, pravastatin, simvastatin, mevastatin, atorvastatin, and cerivastatin, and derivatives, analogs and pharmaceutically acceptable salts thereof, are known as HMG-CoA reductase inhibitors. They are used as antihypercholesterolemic and antihyperlipidemia agents in humans, and are generally produced by fermentation using microorganisms belonging to any one of the Aspergillus, Monascus, Nocardia, Amycolatopsis, Mucor or Penicillium genus. Some of these inhibitors are obtained by treating the fermentation products using the methods of chemical synthesis (as in the case of simvastatin) or they are the products of total chemical synthesis (as in the cases of fluvastatin, atorvastatin and cerivastatin). Some of these are available as a base (such as lovastatin, simvastatin, mevastatin and cervistatin) while others are available as a salt to improve their aqueous solubility (for example, pravastatin atorvastatin and fluvastatin).

HMG-CoA reductase inhibitor stability in an acidic environment is one of a number of problems associated with such compounds, particularly when in the form of pharmaceutically acceptable salt thereof. For example, the degradation kinetics of fluvastatin in an aqueous solution at various pH are illustrated in FIG. 1.

FIG. 1 illustrates the instability of fluvastatin, and by analogy other HMG-CoA reductase compounds, which is directly related to the acidity of the surrounding environment. This instability is due to the extreme lability of the beta and delta-hydroxy groups on the heptenoic acid chain and the presence of a double bond, such that at neutral to acidic pH, the compounds readily undergo any one of elimination, isomerization or oxidation reactions to form conjugated unsaturated aromatic compounds, as well as threo isomers, corresponding lactones, and other degradation products.

U.S. Pat. No. 5,356,896 to Kabadi et al. discloses a stabilized pharmaceutical composition for HMG-CoA reductase inhibitors which is achieved by maintaining an alkaline environment at the site of dissolution in order to stabilize the pharmaceutical composition, so that the aqueous dispersion of the pharmaceutical formulation reaches a pH of at least 8, preferably at least 9 and up to about 10. This was achieved by adding a basifying agent, such as magnesium oxide, which imparts a pH above 9 to an aqueous dispersion of the formulation of the active substance.

However, the local alkaline environment created by the basifying agent at the site of dissolution of the pharmaceutical composition has a negative effect on gastric mucosa. The negative effect is particularly evident for patients with a damaged gastric mucous membrane where the mucosa is not able to create a sufficient acidic environment inside the stomach for normal digestive functioning. This is particularly important in chronic therapies involving HMG-CoA reductase inhibitors.

Additionally, the Kabadi patent also utilizes an enteric coating for its pharmaceutical preparation using materials that are acidic in nature and require special care to employ, such as a barrier coat with a neutral layer underneath the enteric coating. Furthermore, the enteric coating requires a fluid bed coater, which is expensive, requires highly skilled technology and knowledge and is time consuming to operate.

As a way of avoiding the negative effect of localized alkaline environments while stabilizing HMG-CoA reductase inhibitors, U.S. Pat. No. 6,680,341 to Kerc disclosed that HMG-CoA reductase inhibitors could be protected from pH-related destabilization by the introduction of a buffering agent to the active ingredient. The buffering agent creates a resistance to a change in the pH level of the local environment at the site of dissolution. This resistance is created by way of ion exchange between the base ions of the buffering agent and the acid ions present at the site of dissolution causing the neutralization of the acid ions rather than permitting the acid ions to destabilize the active ingredient.

However, the presence of an artificially increased amount of buffering agent in the gastric system disrupts the body's natural regulatory changes in pH. Such disruption negatively affects the absorption of drugs in the body.

Therefore, in order to achieve suitable dosage forms comprising HMG-CoA reductase inhibitors, it is desirable to adequately protect the active ingredient against pH-related destabilization while avoiding the negative effects of localized alkaline environments at the site of dissolution, the negative effects of an artificially increased amount of buffering agents in the gastric system and avoiding the additional problems presented by the use of an enteric coating.

Additionally, the heat and light sensitivity, as well as the hygroscopicity of the subject compounds impose particular requirements in the manufacture and storage of pharmaceutical dosage forms, such as specialized moisture protective packaging materials. It is desirable to minimize the problems associated with such manufacture and storage.

### SUMMARY OF THE INVENTION

The present invention is a pharmaceutical composition containing an HMG-CoA reductase inhibitor as an active ingredient, which is protected from destabilization in acidic environments while avoiding the aforementioned negative effects.

The present invention further provides a process for the preparation of a pharmaceutical composition containing an HMG-CoA reductase inhibitor as an active ingredient which is protected from destabilization in acidic environments while avoiding the aforementioned negative effects.

The present invention further provides a pharmaceutical composition and a process for its preparation, containing an HMG-CoA reductase inhibitor as an active ingredient which is protected from destabilization in acidic environments while avoiding the additional problems presented by the use of an enteric coating and by the manufacturing and storage requirements.

According to a preferred embodiment of the present invention, there is provided a pharmaceutical composition containing an HMG-CoA reductase inhibitor as an active ingredient which is stabilized in acidic and other environments. The stabilization of degradation of an HMG-CoA reductase inhibitor is achieved by maintaining a pH above 7.0 for the active ingredient and by protecting the active ingredient against acidic degradation without using any alkaline medium or buffering agents.

According to another preferred embodiment of the present invention, there is provided the pharmaceutical composition containing an HMG-CoA reductase inhibitor which is protected from premature degradation in the gastric region by utilizing beta-cyclodextrin as an inclusion complexing agent which prevents the dissociation of basic and acidic ions of the molecule of the active ingredient contained within its cavity once the molecule is encapsulated therein.

According to another preferred embodiment of present invention, there is provided the process for the preparation of a pharmaceutical composition containing an HMG-CoA reductase inhibitor as an active ingredient which is stable in acidic and other environments, overcoming the hygroscopic nature of HMG-CoA reductase inhibitors, thereby providing for the composition in a free flowing powder form aiding in encapsulation and tablet formation without the need for enteric coating or the maintenance of sensitive environmental conditions.

According to another preferred embodiment of the present invention, there is provided a pharmaceutical composition comprising an HMG-CoA reductase inhibitor, a cyclodextrin, a lubricant and a filler.

According to another preferred embodiment of the present invention, there is provided a method of preparing pharmaceutical compositions which comprises mixing an HMG-CoA reductase inhibitor compound and a cyclodextrin with water to form a slurry, drying the slurry and mixing the dried slurry with a filler and a lubricant and encapsulating the resulting mixture to form capsules capable of delivering the active ingredient.

Other features and advantages will be apparent from the specification and claims that describe the invention.

### BRIEF DESCRIPTION OF THE DRAWINGS

FIG. 1 is a table indicating the degradation kinetics of fluvastatin in an aqueous solution at various pH levels.

FIG. 2 is a diagram illustrating the chemical structure of beta-cyclodextrin.

FIG. 3 is a diagram illustrating the complexation of drugs inside the hydrophobic cavity of beta-cyclodextrin.

FIG. 4 is a diagram illustrating the equilibrium process describing the interaction between a cyclodextrin and an insoluble drug molecule to form a soluble or insoluble complex.

FIG. 5 is a graph illustrating the correlation between the percentage of complexed drug and cyclodextrin concentration at various K values.

FIG. 6 is a table showing the dissolution of sample capsules as compared to a control

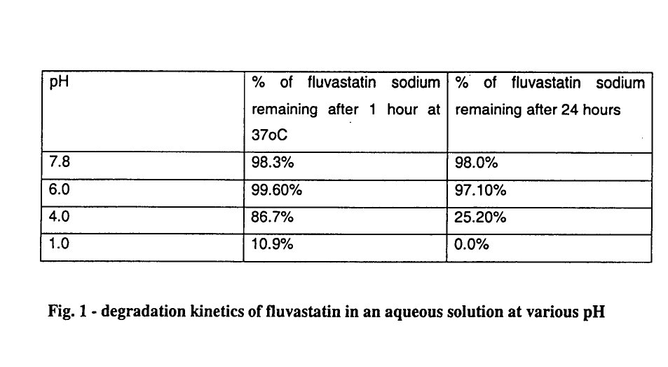

### DETAILED DESCRIPTION OF THE PREFERRED EMBODIMENTS

Pharmaceutical compositions containing an HMG-CoA reductase inhibitor that are stable at basic pH levels, that is, pH greater than 7.0, are known. It is also known that higher pH levels yield more stable pharmaceutical compositions containing an HMG-CoA reductase inhibitor and that a pH of at least 8, and more preferably a pH greater than 9, is desired. However, the acidic environment of the stomach rapidly lowers the pH of a pharmaceutical composition containing an HMG-CoA reductase inhibitor to a point below which destabilization occurs.

In the present invention, pharmaceutical compositions containing an HMG-CoA reductase inhibitor are protected against destabilization in the acidic environment of the stomach by utilizing beta-cyclodextrin as an inclusion complexing agent.

Complexation is one way to favorably enhance the physicochemical properties of pharmaceutical compounds. It is loosely defined as the reversible association of a substrate and ligand to form a new species. Although the classification of complexes is somewhat arbitrary, the differentiation is usually based on the types of interactions and species involved, e.g., metal complexes, molecular complexes, inclusion complexes, and ion-exchange compounds. Cyclodextrins are classic examples of compounds that form inclusion complexes. These complexes are formed when a “guest” molecule is partially or fully included inside a “host” molecule with no covalent bonding. When inclusion complexes are formed, the physicochemical parameters of the guest molecule are disguised or altered, and improvements in the molecule's solubility, stability, taste, safety and bioavailability are commonly seen.

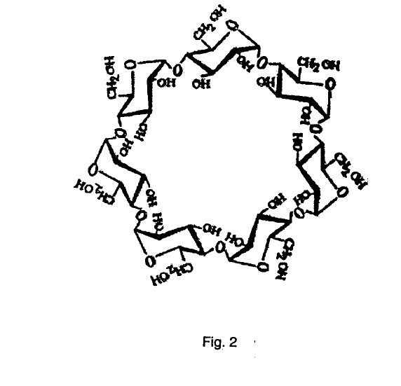

Numerous cyclodextrins with different complexing abilities are available. Cyclodextrins are cyclic oligosaccharides containing 6, 7, or 8 glucopyranose units, referred to as alpha, beta or gamma cyclodextrin, respectively. Each glucose unit contains two secondary alcohols at C-2 and C-3, and a primary alcohol at the C-6 position, providing 18-24 sites for chemical modification and derivatization. The chemical structure of beta-cyclodextrin is shown in FIG. 2.

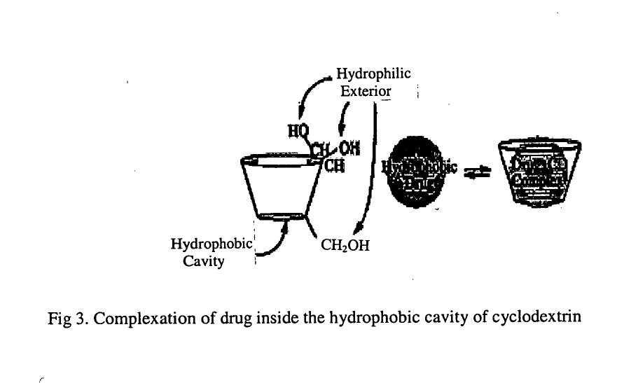

The 3-dimensional structure of the parent cyclodextrin provides a cavity, as shown in FIG. 3, which is hydrophobic relative to an aqueous environment. The sequestration of hydrophobic drugs inside the cavity of the cyclodextrin can improve the drug's solubility and stability in water, the rate and extent of dissolution of the drug:cyclodextrin complex, and the bioavailability of the drug when dissolution and solubility are limiting the delivery. These cyclodextrin properties enable the creation of formulations for insoluble drugs which are typically difficult to formulate and deliver with more traditional excipients.

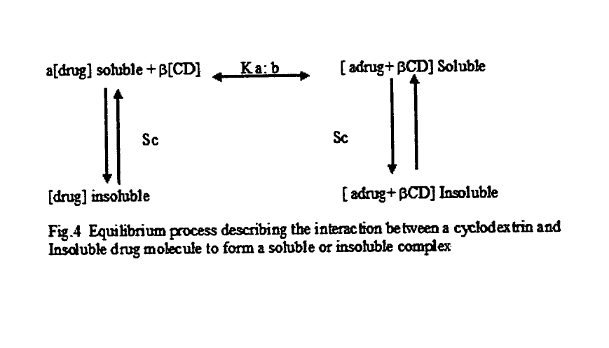

Cyclodextrins form inclusion complexes with hydrophobic drugs through an equilibrium process, FIG. 4, quantitatively described by an association or stability constant (K_{a:b}), where

$${K}_{a \text{:} b} = \frac{[ {Drug}_{a} {CD}_{b} ]}{{{[ Drug ]}^{a} [ CD ]}^{b}}$$

where a and b represent the molar ratio of the sequestered drug molecule to the cyclodextrin. The magnitude of this associate constant can be used to compare the binding effectiveness of different cyclodextrins. Various complexes with different ratios of drug-to-cyclodextrin molecules can be formed, depending on the type of cyclodextrin used and the size and physicochemical characteristics of the drug molecule. In dilute solutions, or if the drug fits entirely into the cyclodextrin cavity, a 1:1 complex results. However, if the cavity is large enough, two drug molecules may be accommodated, resulting in the formation of a 2:1 complex. Conversely, if the drug is very large, then more than one cyclodextrin molecule might enclose the drug for the formation of 1:2 or higher order complexes. Although each complex has a finite stoichiometry, more than one complex may be formed in a given system. Depending on the method used to determine the association constant, it is possible to obtain a description of the stoichiometry of the complex (a:b).

Complexation of drugs by cyclodextrins improves their delivery characteristics and does not interfere with their activity because complexation is a rapidly reversible process. In an aqueous solution, drug:cyclodextrin complexes are continually forming and dissociating with lifetimes in the range of milliseconds or less. Although slower dissociation kinetics are seen with stronger binding, the rates are still essentially instantaneous.

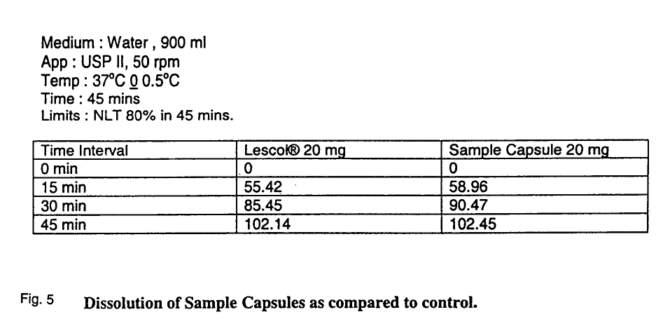

The inclusion complex is resistant to hydrolysis in the acidic environment of the stomach, thus maintaining the active ingredient as a guest within the inclusion complex following oral administration and permitting the active ingredient to pass through the stomach without degradation and destabilization. However, the inclusion complex is not resistant to digestion by enzymes present in the intestinal region, thus causing its breakdown and the release of the active ingredient for absorption. In some cases, the drug is released from the inclusion complex upon dilution with contributions from competitive displacement with endogenous lipophiles binding to plasma and tissue components where drug uptake into tissues is not available to the complex and the beta-cyclodextrin is rapidly eliminated. The effects of dilution are demonstrated in FIG. 5 for complexes with various binding constants. Most drug:cyclodextrin complexes exhibit binding constants in the range of 100-20,000 M¹ and even for the more tightly bound drugs, a 1:100 dilution will reduce the percentage of complexed drug from 100% to 30%. A 1:100 dilution is readily attainable for intravenous products, and upon dilution in the stomach and intestinal contents.

Dilution is minimal, however, when drugs are administered via other routes such as ophthalmic, transmucosal, and transdermal. Under these conditions, the drug can still be displaced from the cyclodextrin cavity by competing lipophiles, such as triglycerides, cholesterol, bile salts, and other hydrophobic compounds often found in high concentrations at the site of delivery.

With respect to orally administered dosage forms, in order to protect the active ingredient from degradation in the acidic environment of the stomach, an inclusion complex is formed with beta-cyclodextrin and the active ingredient by creating a slurry with beta-cyclodextrin in water, which forms the cavity structure as seen in FIG. 3. The active ingredient, which is capable of providing a stable pH greater than 7 and preferably greater than 8, is sequestered inside the cavity upon drying. While sequestered inside the cavity, the active ingredient is protected from degradation in the acidic environment in the stomach due to beta-cyclodextrin's resistance to acidic hydrolization. This allows the composition to pass through the stomach in a stable form to be released in the intestines due to beta-cyclodextrin's affinity to hydrolization by enzymatic processes.

The active ingredient of the present invention is an HMG-CoA reductase inhibitor, which can be any one of the group of fluvastatin, lovastatin, pravastatin, simvastatin, mevastatin, atorvastatin, cerivastatin, the derivatives, analogs and pharmaceutically acceptable salts thereof. The formulation of the beta-cyclodextrin and the HMG-CoA reductase inhibitor is mixed with relatively neutral pH excipients that act as dilutents or fillers, such as sorbitol or lactose, to make up the weight required to fill a capsule or tablet, and lubricants, such as magnesium stearate or talc, to promote smooth flow of the mixture. Persons skilled in the art will recognize that other dilutents, fillers and lubricants may be suitable.

The resulting pharmaceutical composition provides an active ingredient that is stable and protected against degradation in the acidic environment of the stomach without creating an alkaline medium or using a buffering agent, thus avoiding the problems created thereby. The resulting pharmaceutical composition also provides an active ingredient that is stable and protected against degradation in the acidic environment of the stomach without the use of enteric coatings, thus avoiding the problems created thereby.

The pharmaceutical compositions according to the present invention may be prepared as described below.

A calculated amount of water is transferred into a vessel with a stirrer into which beta cyclodextrin is slowly mixed in. An HMG-CoA reductase inhibitor is added in small lots to avoid the formation of lumps, and the mixture is stirred until at least homogenization. After homogenization, the mixture is dried. The dried mixture is milled and passed through a mesh. The pH of the composition should be more than 7 and preferably greater than 8 for achieving maximum stability. Using a complexation technique with beta-cyclodextrin prevents the degradation of the active ingredient in the gastric media.

The resulting stabilized composition is then formulated with other excipients, including a filler such as sorbitol, which is freely soluble in water and has a pH between 6.0 and 7.0 in water, and a lubricant, such as magnesium stearate. The final formulation may be prepared in capsule form. The pH of the final composition in water is found to be about 9.4, which provides for stability of the active ingredient inside the dosage form.

Although the foregoing invention has been described in some detail for purposes of illustration, it will be readily apparent to one skilled in the art that changes and modifications may be made without departing from the scope of the invention described herein.

The present invention will be further illustrated by means of the following examples. It is to be understood, however, that the invention is not meant to be limited to the details described therein.

### EXAMPLE 1

### Fluvastatin Capsule 20 mg

The pharmaceutical composition with the active ingredient of fluvastatin sodium in the form of capsules is prepared as follows. Water and beta-cyclodextrin are mixed to create a slurry. Fluvastatin sodium is added to the slurry and stirred over a period of time. The resulting slurry is then dried and milled. The resulting formulation is mixed with sorbitol and magnesium stearate. The resulting mixture is then put into capsules containing 20 mg of fluvastatin sodium.

| | | |
|---|---|---|
| 1. Fluvastatin sodium equivalent to fluvastatin | 21.12 | mg = 20 mg |
| 2. Beta cyclodextrin | 31.68 | mg |
| 3. sorbitol | 131.2 | mg |
| 4. Magnesium stearate | 4.00 | mg |
| 5. Purified water | q.s | |
| | 188.00 | mg/capsule |

### EXAMPLE 2

### Fluvastatin Capsule 40 mg

The pharmaceutical composition with the active ingredient of fluvastatin sodium in the form of capsules is prepared as follows. Water and beta-cyclodextrin are mixed to create a slurry. Fluvastatin sodium is added to the slurry and stirred over a period of time. The resulting slurry is then dried and milled. The resulting formulation is mixed with sorbitol and magnesium stearate. The resulting mixture is then put into capsules containing 40 mg of fluvastatin sodium.

| | | |
|---|---|---|
| 1. Fluvastatin sodium equivalent to fluvastatin | 42.24 | mg = 40 mg |
| 2. Beta cyclodextrin | 63.36 | mg |
| 3. sorbitol | 262.40 | mg |
| 4. Magnesium stearate | 8.00 | mg |
| 5. Purified water | q.s | |
| | 376 | mg/capsule |

Sample capsules containing fluvastatin sodium as the active ingredient were prepared according to the above examples and were subject to in vitro dissolution studies. It was found that the comparative in vitro dissolution of the sample capsules with respect to Lescol®, used as a control, was equivalent, as shown in FIG. 6.

While the invention has been described and illustrated with reference to certain particular embodiments thereof, those skilled in the art will appreciate that various adaptations, changes, modifications, substitutions, deletions, or additions of procedures and protocols may be made without departing from the invention. It is intended, therefore, that the invention be defined by the scope of the claims that follow and that such claims be interpreted as broadly as is reasonable.

What is claimed is:

1. A pharmaceutical composition comprising an HMG-CoA reductase inhibitor compound, a cyclodextrin, a lubricant and a filler.

2. A pharmaceutical composition according to claim 1 wherein the HMG-CoA reductase inhibitor compound has a pH greater than 7.

3. A pharmaceutical composition according to claim 2 wherein the cyclodextrin is chosen from a group consisting of alpha-cyclodextrin, beta-cyclodextrin and gamma-cyclodextrin.

4. A pharmaceutical composition comprising according to claim 3 wherein the HMG-CoA reductase inhibitor compound is fluvastatin sodium and the cyclodextrin is beta-cyclodextrin.

5. A pharmaceutical composition according to claim 1 wherein the percentage weight of the HMG-CoA reductase inhibitor compound is 10 to 11%, and the percentage weight of the cyclodextrin is 15 to 17%.

6. A pharmaceutical composition according to claim 4 wherein the lubricant is magnesium stearate and the filler is sorbitol and wherein the percentage weight of fluvastatin sodium is 10 to 11%, the percentage weight of beta-cyclodextrin is 15 to 17%, the percentage weight of magnesium stearate is 1 to 3% and the percentage weight of sorbitol is 70 to 71%.

7. A pharmaceutical composition according to claim 4 which is suitable for encapsulation for delivering fluvastatin sodium wherein the lubricant is magnesium stearate and the filler is sorbitol and wherein the percentage weight of fluvastatin sodium is 10 to 11%, the percentage weight of beta-cyclodextrin is 15 to 17%, the percentage weight of magnesium stearate is 1 to 3% and the percentage weight of sorbitol is 70 to 71%.

8. A pharmaceutical composition according to claim 7 wherein the fluvastatin sodium is present in a dosage amount selected from the group of 20 and 40 mg amounts.

9. A method of preparing the pharmaceutical composition of claim 1 comprising the steps of:
a) mixing the HMG-CoA reductase inhibitor compound and the cyclodextrin with water to form a slurry;
b) drying the slurry; and
c) mixing the dried slurry with the lubricant and the filler.

10. A method of preparing the pharmaceutical composition of claim 7 comprising the steps of:
a) mixing the fluvastatin sodium and the beta-cyclodextrin with water to form a slurry;
b) drying the slurry;
c) mixing the dried slurry with the sorbital and the magnesium stearate; and
d) encapsulating the resulting mixture to form capsules capable of delivering fluvastatin sodium.

11. A method of preparing pharmaceutical compositions comprising the steps of:
a) mixing an HMG-CoA reductase inhibitor compound and a cyclodextrin with water to form a slurry;
b) drying the slurry; and
c) mixing the dried slurry with a filler and a lubricant.

12. A method of preparing pharmaceutical compositions according to claim 11 wherein the HMG-CoA reductase inhibitor compound has a pH greater than 7.

13. A method of preparing pharmaceutical compositions according to claim 12 wherein the cyclodextrin is chosen from a group consisting of alpha-cyclodextrin, beta-cyclodextrin and gamma-cyclodextrin.

14. A method of preparing pharmaceutical compositions according to claim 13 wherein the HMG-CoA reductase inhibitor compound is fluvastatin sodium and the cyclodextrin is beta-cyclodextrin.

15. A method of preparing pharmaceutical compositions according to claim 14 wherein the lubricant is magnesium stearate and the filler is sorbitol and wherein the percentage weight of fluvastatin sodium is 10 to 11%, the percentage weight of beta-cyclodextrin is 15 to 17%, the percentage weight of magnesium stearate is 1 to 3% and the percentage weight of sorbitol is 70 to 71%.

16. A method of preparing pharmaceutical compositions according to claim 14 which is suitable for encapsulation for delivering a fluvastatin sodium wherein the lubricant is magnesium stearate and the filler is sorbitol and wherein the percentage weight of fluvastatin sodium is 10 to 11%, the percentage weight of beta-cyclodextrin is 15 to 17%, the percentage weight of magnesium stearate is 1 to 3% and the percentage weight of sorbitol is 70 to 71%.

17. A method of preparing pharmaceutical compositions according to claim 16 wherein the HMG-CoA reductase inhibitor compound is present in a dosage amount selected from the group of 20 and 40 mg in amounts.

18. A solid unit dosage formed by the method of claim 16.

19. A method of treating a person having a condition selected from the group consisting of hypercholesterolemia and hyperlipidemia using the solid unit dosage form as claimed in claim 18.

20. The method of treating a person of claim 19, wherein the solid unit dosage form is in capsule form containing the thus prepared pharmaceutical composition.
